(19) United States
(12) Patent Application Publication
(10) Pub. No.: US 2008/0306347 A1
(43) Pub. Date: Dec. 11, 2008

(54) System and Method for a Telephone Feedback System for Fitness Programs

(76) Inventor: Fred Deutsch, New York, NY (US)

Correspondence Address: PAUL, HASTINGS, JANOFSKY & WALKER LLP, 875 15th Street, NW, Washington, DC, 20005, US

(21) Appl. No.: 11/720,064

(22) PCT Filed: Nov. 23, 2005
(86) PCT No.: PCT/US05/42556

§ 371 Date: Jan. 11, 2008

Related U.S. Application Data
(60) Provisional application No. 60/630,771, filed on Nov. 23, 2004.

(51) Int. Cl. A61B 5/00

(52) U.S. Cl. 600/300

(57) ABSTRACT

A diet management system and method thereof is disclosed, which aids in the facilitation of a diet and fitness feedback and tracking. In particular, a communication module, text-to-speech converter, VOX, and server are used in the diet management system. Several communication devices are employed that allows the dieter a broad choice of communicating dietary information. Moreover, the processing and storing of dietary reports are maintained by a network server.

### BACKGROUND

1. Field of the Inventions

The present invention relates to diet and fitness aids and specifically systems and methods for providing real-time feedback to a dieter.

2. Background Information

With the large number of diets and diet products on the market, the dieting market has become a multi-billion dollar industry. With the desire by many to lose weight and get in better shape, dieters seek any advantage to improve their success rate, which has included getting encouragement from family members, loved ones, and support groups. Though encouragement may seem insignificant, a moment of weakness in discipline can spoil a dieter's hard work.

Many diet and fitness programs track and record certain data related to the type and quantity of food being consumed by the dieter. This data typically comprises the type and amount of food being consumed with each meal. From this, the amount of calories, carbohydrates, fats, etc., can be determined, e.g., on a daily, weekly, or monthly basis. This data can be used to give feedback to the dieter as to his progress, however, it is often difficult for a dieter to keep track of their food intake and to calculate the amount of calories, carbohydrates, fat, etc., consumed. For example, a dieter may be called to an impromptu dinner with a business client. In such a circumstance, it may be inconvenient or awkward for the dieter to record his food intake and calculate his food statistics. Furthermore, it may be especially difficult to keep track of in-between meals and snacks.

In addition to tracking eating habits, data and statistics related to the dieter's food consumption can be used to make adjustments to the dieter's diet and/or exercise regime. For example, if in a particular week, the dieter's intake of calories or carbohydrates exceeds the dieter's allotted amount, the dieter's exercise regime can be increased to compensate for the surplus. If the types of food the dieter is consuming result in an excess of calories, alternative foods can be suggested to meet the calorie requirements of the dieter.

### SUMMARY OF INVENTION

The present invention provides a feedback management system that tracks and records a dieter's eating habits and uses this information to calculate and feedback food intake back to the dieter. In one aspect of the invention, a diet management system for providing feedback to a dieter comprises a communication module configured to receive dietary information from a dieter; a server configured to store the dietary information and provide dietary reports in response to a dieters request.

In one embodiment, a diet management method for providing feedback to a dieter comprising the steps of receiving dietary information from the dieter and communicating the dietary information to a server; storing the dietary information and processing dietary reports in response to a dieter's request by the server.

### BRIEF DESCRIPTION OF THE DRAWINGS

For a better understanding of the nature of the features of the invention, reference should be made to the following detailed description taken in conjunction with the accompanying drawings, in which:

FIG. 1 is a diagram illustrating a diet management system in accordance with one embodiment, and

FIG. 2 is a flowchart illustrating an example method for tracking dietary data and providing feedback to the dieter using the fitness management system of FIG. 1.

### DETAILED DESCRIPTION OF THE INVENTION

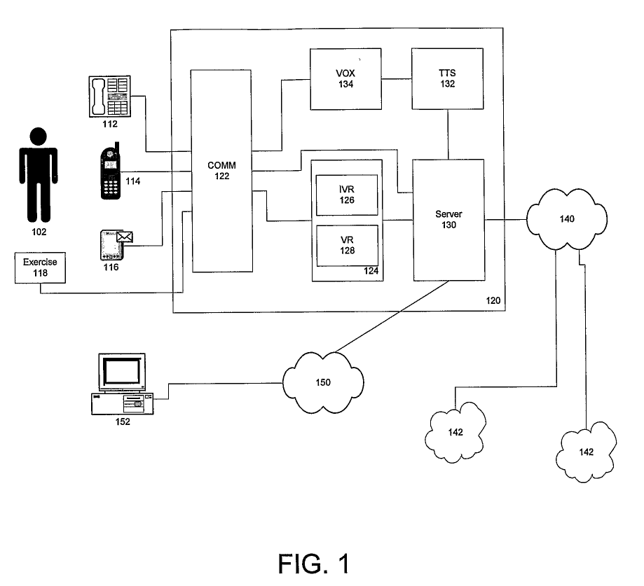

FIG. 1 depicts a diet management system 120, in accordance with one embodiment of the systems and methods described herein, for managing a fitness regime. Dieter 102 can interact with system 120 to provide dietary information to system 120. For example, he can call in using telephone 112, which can be either a standard land line or a cellular phone; he can send a short message system (SMS) message or a text message using wireless device 114; or he can send an email message using any sort of email appliance 116 including a personal computer or a wireless email device, such as a Blackberry. In general, any device or communication method can be used to provide dietary information to system 100. But as explained below, it can be preferable to use a voice communication device, such as a telephone or cellular phone.

In one embodiment, dieter 102 calls system 120 and informs the system of what dieter 102 has or is about to eat. For example, dieter 102 can call system 120 using a telephone or cellular phone and simply tell system 120 what dieter 102 had for breakfast. System 120 can process this information to determine dietary relevant data, such as the amount of calories consumed at breakfast. System 120 can then be configured to provide feedback to dieter 102, e.g., via one of the communication methods described above. For example, system 120 can be configured to provide dieter 102 with voice feedback. In other words, once dieter 102 informs system 120 of what he ate for breakfast, a voice generated by system 120 can tell dieter 102 how many calories he has consumed, how many calories he has left for the day, whether he has consumed too many carbohydrates, etc. In this manner, dieter 102 can get instantaneous feedback on his diet and eating habits. Thus, dieter 102 can constantly maintain awareness of dietary needs, restrictions, and limitations without the overhead normally associated with conventional diets, e.g., measuring, calculating, etc.

In order to interpret dieter 102's vocal response, system 120 can comprise a voice conversion system 124 adapted to convert the dieter's voice information into data that can be processed by a voice processing server 130. Voice conversion system 124 can also comprise a interactive voice response (IVR) module 126 and/or a voice recognition (VR) module 128 in order to communicate with dieter 102. If dieter 102 elects to report his food intake or to receive feedback through a text message or an email message, voice conversion system 124 may be bypassed as required.

Messages to dieter 102 can be converted to speech through a text-to-speech (TTS) module 132. The synthetic speech generated by TTS module 132 can be morphed into voice using a voice morphing module 134. With the speech converted, dieter 102 can receive the message on, e.g., telephone 112. If the response is an immediate response to information provided by dieter 102, the response can be given during the same telephone call that dieter 102 initiated; however, in some circumstances, system 130 can be configured to place a call to dieter 102 and deliver a voice message at a later time.

Server 130 can also be configured to record the processed, dietary data. This stored data can then be used to track the dieter's food intake and check it against certain dietary requirements, which can also be stored on server 130, or within system 120. System 120 can also store, or have access to, fitness information such as a fitness routine or schedule. For example, system 120 can be configured to perform queries to external fitness resources 142 through network 140, which can be a public computer network, private network or virtual private network, or can gather information from its own internal databases and knowledge bases. Based either on its own internal information or information provided by external fitness resources 142 an appropriate course of action can be determined.

In addition, server 130 can comprise a web server which enables dieter 102 to configure his dietary and/or fitness program, review his progress, and receive progress reports through computer 152, which can be connected to system 120 through a computer network 150 such as the Internet.

In order to integrate exercise data with the dietary data stored on system 120, exercise appliance 118 can be coupled to system 120 through a computer network. Exercise appliance 118 can include a wireless heart monitor or a piece of home gym equipment, e.g., that is Internet compatible. In another embodiment, exercise appliance 118 can be coupled to a gym network configured to monitor the progress of ones training regime. Many gyms are equipped with systems which monitor the duration and types of exercise for cardiovascular exercises and weights and repetition of weight training exercises. In addition, a heart rate monitor is often built into the exercise equipment and can be coupled to the network. This data can be transferred from the gym to system 120 either formatted by the gym's system or in raw form from the individual equipment.

Thus, dieter 102 can call in or message in to system 120. System 120 can then ask the dieter what they are eating. Upon the dieter's response, system 120 can calculate and report various facts about the food being consumed including the calorie count, fat count, protein count and carbohydrate count. System 120 can also calculate and report whether dieter 102 is over or under his daily limit and if the choice of food is particularly unhealthy, system 120 can suggest an alternative. All this information can be further packaged so that dieter 102 can view the results later on a web page. As mentioned, in immediate response to dieter 102's food intake report, system 120 can synthesize a response using the voice response of dieter 102's choice.

In one embodiment of system 120, external fitness resources 142 can include online fitness or dietary resources such as ediets or weightwatchers. In such instances, the voice synthesized by voice morphing module 134 can be the voice of the spokesperson of that external fitness resource. This can be used to reinforce the branding of the particular external fitness resource.

In another embodiment of system 120, the voice synthesized by voice morphing module can be configured to report in a foreign language. For example, the voice morphing module can be used to report to dieter 102 results in his native language. Moreover, dieter 102 can, depending on the embodiment, be allowed to select a voice for the reports. For example, dieter 102 can select a famous voice to provide feedback. The famous voice can be translated into any language using voice morphing module 134. Appropriate use of the voice morphing module 134 can accommodate the desires of a wide variety of languages with a wide selection of voices. The voice selected and synthesized by voice morphing module 134 can also be that of a friend or family member of dieter 102. In such embodiments, voice morphing module 134 can be trained based on voice samples received from the selected individual. With a familiar voice, dieter 102 can be given encouragement in maintaining dietary discipline and be dissuaded from taking up bad habits.

In another embodiment of system 120, server 130 or external fitness resource 142 can track dieter 102's eating habits. During the danger periods, that is, periods where dieter 102 is likely to snack or eat improperly, system 120 can be configured to call dieter 102 and present a message to discourage the improper eating.

It should be noted that though FIG. 1 illustrates a system with a variety of modules and functions, embodiments of the system can comprise a subset of the components shown in FIG. 1 and/or a subset of the functionality described above. Furthermore, though components are depicted as separate, many of the modules can be implemented within server 130. Also, the internal databases and knowledge bases can reside within system 120 or reside outside system 120. Therefore, system 120 should not be limited to that architecture shown in FIG. 1 as variations and combinations are obvious to one skilled in the art.

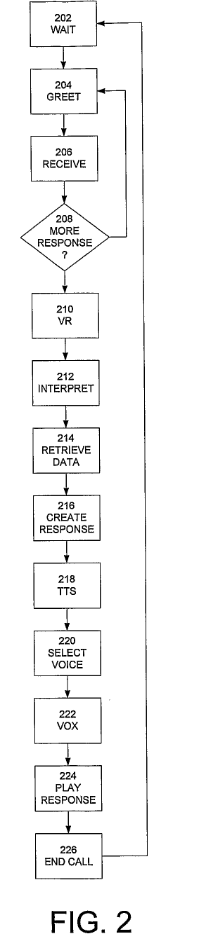

FIG. 2 is a flowchart showing exemplary operation of system 120 in implementing a basic function of system 120. At step 202, system 120 waits for a phone call from dieter 102. When a call is received, system 120 greets dieter 102 at step 204 and prompts dieter 102 for what he is currently eating or for what his is about to eat. At step 206 system 120 receives a voice response from dieter 102. At step 208, a determination is made as to whether any more information is required from dieter 102. This can be based on the context of the information already received such as “I had a ham sandwich, cole slaw AND a pickle for lunch.” In this context, the “and” can indicate all information desired has been reported by dieter 102. Alternatively, step 204 can include a “Is that everything?” prompt and step 206 can comprise an appropriate an affirmative or negative response. In another embodiment, an IVR system is used and dieter 102 can indicate when he is finished by pressing a key on his telephone.

At step 210, the dieter's response can be recognized with VR response module 128. At step 212, the converted data can be parsed and interpreted. In the example given above, “ham sandwich,” “cole slaw” and “pickle” are extracted from dieter 102's response. At step 214, the relevant data is retrieved from either an external fitness resource or from the internal database, which can include, e.g., calorie, carbohydrate, fat and protein statistics about the foods. In addition, dieter 102's profile can be retrieved so information about earlier meals in the day and the type of program dieter 102 is participating in can be factored into a response. At step 216, server 130 determines the appropriate response, for example, it can be a recitation of statistics of the food, dieter progress or a full report. It can also include admonishment for eating poorly or words of encouragement. This response is converted to speech at step 218 using TTS module 132. At step 220, the desired voice and language is selected, which can be retrieved from dieter 102's profile. At step 222, VOX module 134 morphs the speech response from step 218 into the desired language and voice. The resultant response is then played for the caller at step 224. At step 226, the call is ended and system 120 returns to the wait step at step 202.

While certain embodiments of the inventions have been described above, it will be understood that the embodiments described are by way of example only. Accordingly, the inventions should not be limited based on the described embodiments.

What is claimed:

1. A diet management system for providing feedback to a dieter comprising:
a communication module configured to receive dietary information from said dieter; a server configured to store said dietary information and provide dietary reports in response to a dieters request.

2. A diet management system of claim 1, wherein said communication module receives said dietary information in the form of a voice or text message from dieter.

3. A diet management system of claim 2, wherein said the communication module includes a Voice to Text converter for translating voice messages received from the dieter into the stored dietary information.

4. The diet management system of claim 1, wherein the communication module is configured to receive exercise information for a dieter.

5. The diet management system of claim 1, wherein the server is configured to update the dieter regarding the dieter's diet progress.

6. The diet management system of claim 5, wherein the server provides diet updates by sending an electronic message to the dieter

7. The diet management system of claim 5, wherein the server provides diet updates by initiating a phone call with the dieter.

8. The diet management system of claim 4, wherein the server provides instantaneous reports to a dieter based on a combination of the dieter's stored dietary information and stored exercise information.

9. A diet management system of claim 8, wherein the dietary reports is converted to voice through said TTS module and communicated to the dieter by telephone or cellular phone.

10. A diet management system of claim 9, wherein the stored dietary information is accessible by the dieter through a computer network.

11. A diet management system of claim 10, wherein the server comprises a web server that is connected to the Internet.

12. A diet management system of claim 9, wherein the voice is synthesized and morphed for reinforcement and branding of a particular external fitness resource.

13. A diet management system of claim 12, wherein the voice is synthesized and morphed to report into a foreign language.

14. A diet management method for providing feedback to a dieter comprising the steps of:
receiving dietary information from the dieter and communicating the dietary information to a server;
storing said dietary information and processing dietary reports in response to a dieter's request by the server.

15. A diet management method of claim 14, wherein said method further comprises the step of:
converting the report into voice using a Text-to-Speech (TTS) module.

16. A diet management method of claim 15, wherein said method further comprises the step of:
synthesizing said converted voice response using a VOX module.

17. A diet management method of claim 16, wherein said method further comprises the step of:
playing back the synthesized voice response to said dieter.

18. A diet management method of claim 14, wherein said method further comprises the step of:
synthesizing said voice to report in a foreign language, wherein said step is performed after said TTS conversion.

19. A diet management method of claim 14, wherein said method further comprises the step of:
sending real-time dietary feedback to said dieter.

20. A diet management method of claim 14, wherein said method further comprises the step of:
communicating said voice response to the dieter by telephone, cellular phone or text messaging device.
